(19) United States
(12) Patent Application Publication
(10) Pub. No.: US 2019/0030113 A1
(43) Pub. Date: Jan. 31, 2019

(54) DIPEPTIDYL ALDEHYDES FOR THE TREATMENT AND/OR PREVENTION OF PARASITIC DISEASES

(71) Applicant: AXIAVA PHARMA UG, Wuppertal (DE)

(72) Inventors: Jacobus Constantinus Antonius van Meel, Mödling (AT); Bartholomeus Stephanus Josep van Toor, Zürich (CH)

(21) Appl. No.: 16/077,030

(22) PCT Filed: Feb. 8, 2017
(86) PCT No.: PCT/EP2017/052775

§ 371 Date: Aug. 9, 2018

(30) Foreign Application Priority Data
Feb. 10, 2017 (EP) 16155084.3

(51) Int. Cl. A61K 38/05; A61P 33/02

(52) U.S. Cl. CPC A61K 38/05; A61K 31/541; A61K 2300/00; A61P 33/02

(57) ABSTRACT

Several parasites responsible for mammalian diseases are dependent on cysteine proteases for various life-cycle functions. Inhibition or decreasing function of specific cysteine proteases can be useful in the treatment and/or prevention of these parasitic diseases including trichomoniasis, histomoniasis, coccidiosis, trypanosomiasis (trypamonosis) and cryptosporidiosis. Compounds of formula I of the invention are capable of treating and/or preventing the above-identified diseases in mammals, e.g. in avian species as Galliformes including chickens, turkeys, ducks, geese, grouse, guinea fowl, peafowl, quail, partridges, and pheasants, Falconiformes, Passiformes, Columbiformes (i.e. Columba livia domestica), Psittaciformes, and in domestic mammals such as cattle, sheep, goats, horses, pigs, camels, lamas, alpacas, cats and dogs.

### FIELD OF THE INVENTION

The present invention relates to dipeptidyl aldehyde compounds of formula I and their use for treating and/or preventing parasitic diseases including trichomoniasis, histomoniasis, coccidiosis, trypanosomiasis and cryptosporidiosis.

### BACKGROUND ART

Several parasites responsible for mammalian diseases are dependent on cysteine proteases for various life-cycle functions. These proteases which are crucial to the activities of parasitic organisms are primarily categorized into cathepsin B-like and cathepsin L-like. These enzymes figure prominently in various host-parasite interactions, such as evasion of host immune attacks, adaptation to the host, and tissue invasion. Therefore it has been hypothesized that specific inhibitors of cathepsin L-like proteases including cruzain (=Trypanosoma cruzi cathepsin L cysteine protease) may be regarded as potential new targets for antiparasitic therapeutics. Recently it has been found that Trichomonas gallinae secretes certain cysteine proteases which may play a crucial role in the cytopathogenic effects to tissues of the host (1). Amin et al (2012) described that cysteine peptidases, secreted by Trichomonas gallinae, are involved in the cytopathogenic effects on a permanent chicken liver cell culture. These observations led to the hypothesis that specific inhibitors of cysteine proteases can be of therapeutic value in the treatment and/or prevention of several parasitic diseases, among these are trichomoniasis, histomoniasis, coccidiosis, trypanosomiasis and cryptosporidiosis.

Trichomoniasis is caused by Trichomonas gallinae, a single-celled, pear-shaped protozoan with 4 whip-like anterior flagella and a fin-like undulating membrane that extends for approximately ⅔ of the total body length. T. gallinae is a parasite of the upper digestive tract that affects many avian species and causes accumulation of necrotic material in the mouth and esophagus. It is principally a disease of young birds and is often fatal. The disease has been found in domestic or common pigeons, doves, quails, turkeys, chickens, falcons, hawks, various finches, the Java sparrow, and canaries. Trichomoniasis is also known as Canker (in doves and pigeons) and as Frounce (in raptors). Treatment is only feasible in captive birds because the drugs used for treatment must be administered orally, either by force feeding or by treating the food and/or water. Antiprotozoal medications that have been used are among others dimetridazole (50 mg/kg body weight, or in the drinking water at 0.05% for 5-6 days), metronidazole (60 mg/kg body weight), copper sulfate, quaternary ammonia, carnidazole and ronidazole (20 mg/kg, or 1 g/L drinking water for 10 birds during 3-5 days). Unfortunately resistance against these nitro imidazole types of antiprotozoal agents (i.e. metronidazole, dimetridazole, carnidazole, ronidazole) has been described and this is regarded as a major clinical problem. The aforementioned data indicate a need for new therapeutics to prevent and/or treat trichomonads infections.

Coccidiosis is a highly infectious and very common disease caused by the protozoan Coccidia (Coccidiasina) that infects the intestines of gallinaceous birds including pigeons, chickens, turkeys, ducks, geese, grouse, guinea fowl, peafowl, quail, partridges, and pheasants. Infections in domestic pigeons are typically mixed Eimeria spp. and commonly include Eimeria columbarum, Eimeria columbae and Eimeria labbeana. The reported prevalence of infection is 5.1%-71.9%, and worldwide mortality in juvenile pigeons varies from 5% to 70%, with most deaths occurring in the third and fourth month of life. Chemotherapeutic options used include amprolium, sulfonamides, clazuril, and toltrazuril. Reasons to use toltrazuril include the growing resistance against other drugs, such as sulfonamides and amprolium. Pigeons treated with toltrazuril (Baycox, 20 mg/kg body weight) up to 14 days before the experimental infection showed on average a reduction of more than 97% in the number of oocysts in individual fecal samples. Symptoms of Coccidiosis include little or no desire to eat or drink. Pigeons with Coccidiosis will remain puffed up on perches, and they lack any desire to move, often closing their eyes. Droppings are usually very loose, greenish in color, and may become very watery. Weight loss is another symptom, and death can occur in young birds. The presence of coccidia in pigeons contributes to a reduction in the overall resistance, what causes that the animals are more susceptible to other infections.

Histomonas meleagridis is a species of parasitic protozoan that infects a wide range of gallinaceous birds including chickens, turkeys, ducks, geese, grouse, guinea fowl, peafowl, quail, partridges, and pheasants, causing blackhead disease, infectious enterohepatitis, or histomoniasis. H. meleagridis is the causal organism of histomoniasis of gallinaceous birds. It induces extensive and severe necrosis of the tissues of the mucosa and submucosa of cecum and parenchyma of the liver. The lesions are sometimes exacerbated by other pathogens such as Escherichia coli and coccidia. There are currently no therapeutic drugs prescribed for the disease. Therefore, prevention is the sole mode of treatment. The only drug used for the control (prophylaxis) in the USA is nitarsone (4-nitrophenylarsonic acid) at 0.01875% of feed until 5 days before marketing. This agent is not approved for use in the EU. However, it has been argued that nitarsone similarly as roxarsone may lead to unacceptable concentrations of inorganic arsene in poultry meat which may be harmful to human consumers (2). Nifurtimox (NFX), a compound with known antiprotozoal activity, was demonstrated to be effective at 300-400 ppm, and tolerated by turkeys. However experimental toxicity studies with Nifurtimox evidenced neurotoxicity, testicular damage, ovarian toxicity, and deleterious effects in adrenal, colon, oesophageal and mammary tissue. Additionally significant mutagenic effects and tumorigenic or carcinogenic effects in some studies were shown.

Cryptosporidium is a microscopic parasite that causes the diarrheal disease cryptosporidiosis. Both the parasite and the disease are commonly known as “Crypto”. There are many species of Cryptosporidium that infect humans and animals. The parasite is protected by an outer shell that allows it to survive outside the body for long periods of time and makes it very tolerant to chlorine disinfection. While this parasite can be spread in several different ways, water (drinking water and recreational water) is the most common method of transmission.

In mammals, there are descriptions of infection by C. bovis, C. canis, C. felis, C. meleagridis, C. parvum, and the cervine genotype; in birds, the following species and genotypes have been described: C. baileyi, C. galli, C. meleagrndis, C. parvum and the avian genotypes I, II and III. Several species have also been described in humans, such as C. parvum, C. hominis, and some species adapted to animal hosts such as C. canis, C. felis and C. meleagridis.

WO2/048097A1 discloses compounds and pharmaceutical compositions useful as anti-parasitic agents, particularly in the treatment, prevention or amelioration of one or more symptoms of malaria or Chagas' disease. Choe Y. et al (Bioorg Med Chem. 2005 Mar. 15; 13(6):2141-56) disclose α-keto-based inhibitors of cruzain for the treatment of Chagas disease in human people.

Whereas Cryptosporidia are intestinal parasites infecting a variety of animals (e.g. cattle, sheep, rodents, cats and dogs, but also birds, fish and reptiles), human infections occur due to Cryptosporidium parvum, a species that also affects domestic animals. C. baileyi can cause respiratory disease in chickens and turkeys. The same species causes infections of the hindgut and cloacal bursa in chickens, turkeys, and ducks. C. meleagridis also infects both species. A further species causes respiratory disease in quail. The oocysts are excreted ready sporulated in the faeces and infection occurs by inhalation and ingestion. Unfortunately there is currently no known effective treatment in poultry. There are no effective preventative medicines or feed additives.

Trypanosomiasis is a parasitic disease caused by species of flagellate protozoa belonging to the genus Trypanosoma which inhabit the blood plasma and various body tissues and fluids. These parasites are found in many animals but seem to be pathogenic only for mammals, including man. African animal trypanosomiasis can be caused by several species of trypanosomes. T. congolenseis is found in most domestic mammals: cattle, sheep, goats, horses, pigs, camels and dogs, and also in many wild animals. T. vivaxis is a parasite of domestic and wild ruminants and of horses. T. simiaeis is found mainly in domestic and wild pigs. T. bruceiis is a parasite very close to T. gambiense and T. rhodesiense, which are the causes of human sleeping sickness. It can be found in practically all domestic and wild animals. T. evansiis is found in Africa only in the Saharan and Sahelian regions where it is primarily a camel parasite, but it may be a parasite of horses, cattle and dogs as well. It also occurs in Asia—where it commonly causes disease in camels and horses, and less commonly in cattle, water buffaloes, elephants and dogs—and in Central and South America. The trypanocides currently employed are: homidium salts (Ethidium, Novidium), quinapyramine sulfate (Antrycide), diminazene aceturate (Berenil), isometamidium chloride (Samorin, Trypamidium) and suramin sodium sulfonate. However side effects and drug-resistant trypanosomes which are dependent upon the species as well as the used agents, raise considerable problems in the treatment of trypanosomiasis.

Thus there is an urgent need for new, safer, and effective treatment and/or prevention of diseases caused by parasites.

### SUMMARY OF INVENTION

It is the objective of the present invention to provide new therapeutics to treat or prevent parasitic diseases.

The objective is solved by the subject of the present invention.

According to the invention there is provided a compound of general formula I

Z-Phe-X—CHO

wherein
X is tyrosine, methyl tyrosine, butyl tyrosine, alanine, or leucine; and
Z represents N-benzyloxycarbonyl, or
the analogues and derivatives of these dipeptidyl aldehydes or a pharmaceutically acceptable salt or stereoisomer thereof for use in the treatment or prevention of parasitic diseases. Examples of parasitic diseases include trichomoniasis, histomoniasis, coccidiosis, trypanosomiasis and cryptosporidiosis in mammals.

One embodiment of the invention refers to N-(benzyloxycarbonyl)-L-phenylalanyl-L-tyrosinal (Z—FY—CHO, compound b of Table 1, FIG. 1), and further dipeptidyl aldehyde derivatives and analogues with the pharmaceutically acceptable salts and stereoisomers thereof (see Table 1) for use in the treatment or prevention of parasitic diseases.

TABLE 1

| | | cathepsin L (IC₅₀ nM)** |
|---|---|---|
| a | Z-Phe-Phe-CHO | 0.74 |
| b | Z-Phe-Tyr-CHO | 0.85 |
| c | Z-Phe-Leu-CHO | 0.78 |
| d | Z-Phe-Ala-CHO | 15.5 |
| e | Z-Phe-Tyr(Me)-CHO | 2.95 |
| f | Z-Phe-Tyr(Bu)-CHO | 6.96 |

**Data from J-T Woo et al. (4)

Peptidyl aldehyde derivates are potent and selective inhibitors of cathepsin L (4). The compound Z—FY—CHO (N-(benzyloxycarbonyl)-L-phenylalanyl-L-tyrosinal, (CAS 167498-29-5) for instance has an IC₅₀ value of 0.85 nM against cathepsin L cysteine protease. Therefore Z—FY—CHO is specifically valuable.

A further embodiment of the invention relates to a compound for use as described herein, wherein the compound is N-(benzyloxy-carbonyl)-L-phenylalanyl-L-tyrosinal (Z—FY—CHO), or its analogues and derivatives or a pharmaceutically acceptable salt or stereoisomer thereof.

A further embodiment of the invention relates to compounds for use as described herein, wherein the parasitic disease is selected from the group of trichomoniasis, histomoniasis, trypanosomiasis, coccidiosis and cryptosporidiosis.

A further embodiment of the invention relates to a compound for use as described herein, wherein the compound is administered to non-human mammals.

One embodiment of the invention relates to compounds for use as described herein, wherein the non-human mammals are birds, preferably pigeons.

One embodiment of the invention relates to a compound for use as described herein, wherein the compound is administered together with an antiparasitic agent.

One embodiment of the invention relates to compounds for use as described herein, wherein said antiparasitic agent is selected from the group consisting of nifurtimox, arteminisin, suramin, homidium salts (ethidium, novidium), quinapyramine sulfate, diminazene aceturate, isometamidium, metronidazole, dimetridazole, carnidazole, ronidazole, toltrazuril, sulfonamides and amprolium.

A further embodiment of the invention relates to a compound for use as described herein, wherein said antiparasitic compound is administered prior, simultaneously or after administration of the compound.

A further embodiment of the invention relates to a compound for use as described herein, wherein said compound is administered at least once a day for a period of at least 3 days.

A further embodiment of the invention relates to a compound for use as described herein, wherein said compound is administered at least twice a day for a period of at least 3 days.

### BRIEF DESCRIPTION OF DRAWINGS

FIG. 1: Structure of Z—FY—CHO

FIG. 2: Relative mean values of the total T. gallinae trophozoite numbers after 24 hours of treatment with Z—FY—CHO (=compound A in FIG. 2; normalized in relation to the 1% DMSO-treated reference group NCA as percentages; * P<0.05 for reduction compared to group NCA).

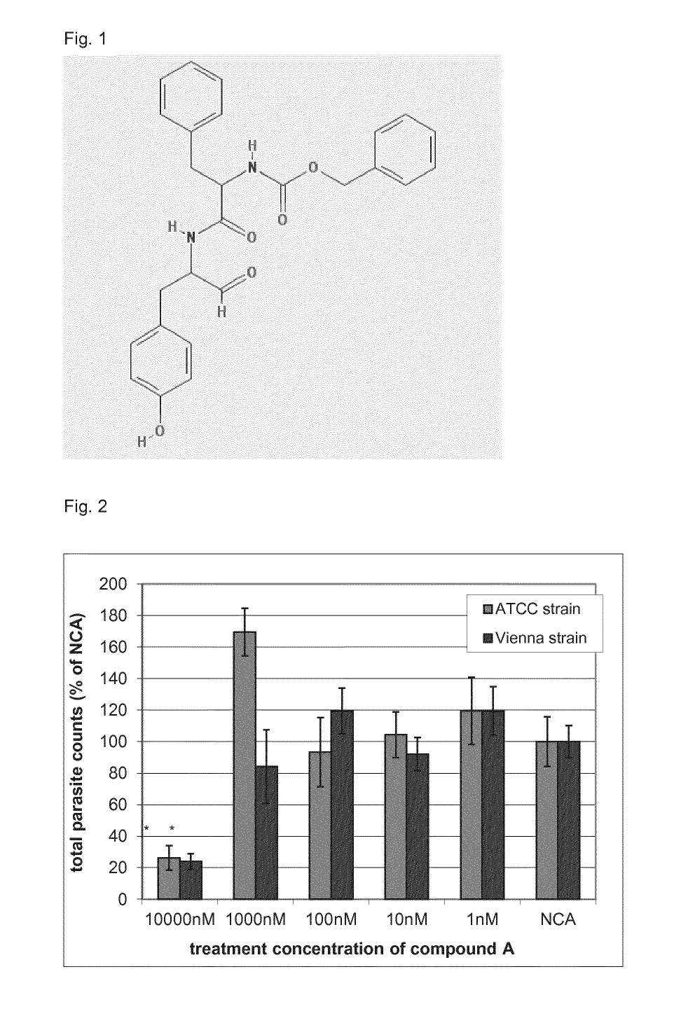

### DESCRIPTION OF EMBODIMENT

Proteases are classified according to their catalytic site into four major classes: serine proteases, cysteine proteases, aspartic proteases and metalloproteases.

Cysteine proteases are proteins with a molecular mass about 21-30 kDa. They show the highest hydrolytic activity at pH 4-6.5. Because of the high tendency of the thiol group to oxidation, the environment of the enzyme should contain a reducing component, glutathione serves as an activating agent in cells, whereas mercaptoethanol or dithiothreitol is required for in vitro experiments. Thus cysteine proteases are generally characterized by the presence of a uniquely reactive thiol that has been shown to catalyze amide bond hydrolysis via a thioester intermediate, this intermediate S-acyl-enzyme moiety is the fundamental step in hydrolysis. Inhibitory activity of compounds can be assessed in vitro by incubation of isolated or recombinant cysteine proteases with substrates such as carbobenzoxy-L-phenylalanyl-L-arginine 4-methyl-coumaryl-7-amide (Z-Phe-Arg-MCA) and monitoring the change in fluorescence intensity which is well known for persons skilled in the art.

The present invention relates to compounds that inhibit or decrease the activity of specific cysteine proteases, namely cruzain and other cathepsin L-like cysteine proteases. Because cathepsin L type cysteine proteases including cruzain are needed for various life-cycle functions of several parasites as for example Trichomonas spcc, Histomonas spcc, Coccidinae spcc, Trypanosoma spcc and Cryptosporidia spcc, compounds of the invention are specifically useful for inhibiting and/or decreasing and/or preventing the growth and/or survival of the abovementioned parasites. Thus, parasitic diseases such as trichomoniasis, histomoniasis, coccidiosis, trypanosomiasis and cryptosporidiosis can be treated and/or prevented by administering compounds of the invention to patients in need thereof.

Additionally, other parasitic diseases caused by parasites that use cruzain or similar cathepsin L-like cysteine proteases for life-cycle functions can also be treated and/or prevented by administration of a compounds of the present invention.

Also included within the scope of the present invention is a pharmaceutical composition which comprises of a compound as described herein and a pharmaceutically acceptable carrier and any of the compounds specifically disclosed in the present application. These and other aspects of the invention will be apparent from the teachings contained herein.

The compounds of the present invention can be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the present invention may be administered in combination with a solubilizer and/or emulsifying agent. Such agents can include for example polyethylene glycol (15)-hydroxystearate (Solutol ©HS 15), polysorbate 80 (Tween 80), Triton X-100, and chremophore.

The compounds of the present invention may be administered in the form of micelles from the class of cyclic oligosaccherides. Such cyclodextrins can include for example α-cyclodextrin, β-cyclodextrin, methyl-β-cyclodextrin, hydroxypropyl-β-cyclodextrin, γ-cyclodextrin and δ-cyclodextrin.

The compounds of the present invention may be coupled with soluble polymers as targetable drug carriers. Such polymers can include for example polyvinylpyrrolidone, pyran copolymer, polyhydroxypropylmethacrylamidephenol, polyhydroxyethylaspartamidephenol, or polyethyleneoxidepolylysine substituted with palmitoyl residues.

The instant compounds are also useful in combination with known agents useful for treating or preventing parasitic diseases, including trichomoniasis, histomoniasis, coccidiosis, trypanosomiasis and cryptosporidiosis.

If formulated as a fixed dose, such combination products employ the compounds of this invention within the dose range described below and the other pharmaceutically active agent(s) within its approved dosage range. Compounds of the instant invention may alternatively be used sequentially with known pharmaceutically acceptable agent(s) when a combination formulation is inappropriate.

The term “administration” and variants thereof (e.g. “administering” a compound) in reference to a compound of the invention means introducing the compound or a prodrug of the compound into the system of the patient in need of treatment. When a compound of the invention or prodrug thereof is provided in combination with one or more other active agents (e.g. a cytotoxic agent, etc.), “administration” and its variants are each understood to include concurrent and sequential introduction of the compound or prodrug thereof and other agents.

The present invention includes within its scope prodrugs of compounds of this invention. In general, such prodrugs will be functional derivatives of the compounds of this invention which are readily convertible in vivo into the required compound. Thus in the methods of treatment of the present invention, the term “administration” shall encompass the treatment of the various conditions described with the compound specifically disclosed or with a compound which may not be specifically disclosed, but which converts to the specified compound in vivo after administration to the patient. Metabolites of these compounds include active species produced upon introduction of compounds of this invention into the biological milieu.

The compounds of this invention may be administered to mammals either alone or preferably in combination with pharmaceutically acceptable carriers or diluents, optionally with known adjuvants in a pharmaceutical composition, according to standard pharmaceutical practice. The compounds can be administered by one or more of the following, orally or parenterally, including intravenous, intramuscular, intraperitoneal, subcutaneous, rectal and topical routes of administration.

In case of tablets for oral use, carriers which are commonly used include lactose and corn starch, and lubricating agents, such as magnesium stearate, is commonly added. For oral administration in capsule form, useful diluents include lactose and dried corn starch. For oral use of a therapeutic compound according to this invention, the selected compound may be administered, for example, in the form of tablets or capsules, or as an aqueous solution or suspension. For oral administration in the form of a tablet or capsule, the active drug component can be combined with an oral, non-toxic, pharmaceutically acceptable inert carrier such as lactose, starch, sucrose, glucose, methyl cellulose, magnesium stearate, dicalcium phosphate, calcium sulphate, mannitol, sorbitol or the like; for oral administration in liquid form, the oral drug components can be combined with any oral, non-toxic, pharmaceutically acceptable inert carrier such as ethanol, glycerol, polyethylenglycol, glycerine, dimethylsulfoxide (DMSO), water and the like. Moreover, when desired or necessary, suitable binders, lubricants, emulsifying (i.e. Tween 80), suspending, and disintegrating agents and coloring agents can also be incorporated into the mixture.

The term “therapeutically effective amount” as used herein is an amount of active compound or pharmaceutical agent that is effective to prevent or slow the development of, or to partially or totally alleviate the existing symptoms in a particular disease, condition or infection for which the subject is being treated (e.g. parasitic disease caused by parasites that rely on cruzain or a similar cathepsin L-like cysteine protease for one or more life-cycle functions such as T. gallinae, E. columbarum, T. cruzi, H. meleagridis or T. congolesis). Determination of a therapeutically effective amount is well within the capability of those skilled in the art.

The terms “treating” or “treatment” of a disease as used herein includes lessening, ameliorating, decreasing and/or inhibiting the disease, e.g., causing the clinical symptoms of the disease or the development of the disease to lessen, decrease, arrest or withdraw; or relieving the disease, e.g., causing regression of the disease or its clinical symptoms.

The term “prevent” or “preventing” of a disease as used herein includes causing the clinical symptoms of the disease not to develop in a mammal that may be exposed to or predisposed to the disease or likely to get the disease (i.e., by travel to or through an affected geographical region or having a genetic predisposition) but does not yet experience of display symptoms of the disease; inhibiting the disease.

The terms “daily for one to seven days” and “once weekly”, as used herein, means that a unit dosage, for example a unit dosage of a compound of the instant invention, is administered at least once day for one to seven days. In the once-weekly dosing regimen, the unit dosage is generally administered about once in every seven days.

Also of interest are any pharmaceutically-acceptable derivatives, including salts, esters, acids, enol ethers and esters, bases, solvates, hydrates and prodrugs of the compounds described herein. Pharmaceutically-acceptable salts, include, but are not limited to, amine salts, such as but not limited to N, N′-dibenzylethylenediamine, chloroprocaine, choline, ammonia, diethanolamine and other hydroxyalkylamines, ethylenediamine, N-methylglucamine, procaine, N-benzylphenethylamine, 1-p-chlorobenzyl-2-pyrrolidin-1′-yl-methyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

In one exemplary application, a suitable amount of compound is administered to a mammal undergoing treatment for a parasitic disease. Oral dosages of the present invention when used for the indicated effects will range between about 1 to 100 mg/kg/day, and most preferably 5 to 30 mg/kg/day. Furthermore, compounds of the present invention can be administered via oral use of suitable vehicles well known to those of ordinary skill in the art.

In another exemplary application, oral dosages of the present invention, when used for the indicated effects, will range between about 10 mg/kg per week to about 700 mg/kg per week, preferably 35 to 250 mg/kg/week.

The compounds of the present invention can be used in combination with other agents useful for treating parasitic diseases. The individual components of such combinations can be administered separately at different times during the course of therapy or concurrently in divided or single combination forms. The instant invention is therefore to be understood as embracing all such regimens of simultaneous or alternating treatment and the term “administering” is to be interpreted accordingly.

The compounds of the present invention may have asymmetric centers, chiral axes, and chiral planes, and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted.

The pharmaceutically acceptable salts of the compounds of this invention include the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic, and the like. The preparation of the pharmaceutically acceptable salts described above and other typically pharmaceutically acceptable salts is more fully described by Berge et al. (3).

### EXAMPLES

The Examples which follow are set forth to aid in the understanding of the invention but are not intended to, and should not be construed to limit the scope of the invention in any way. The Examples do not include detailed descriptions of conventional methods; such methods are well known to those of ordinary skill in the art.

### Example 1—In Vitro Effects on Growth and Viability of T. gallinae

Z—FY—CHO was tested in an in vitro assay on growth and viability of two strains of T. gallinae (Vienna strain and ATCC 30095). T. gallinae is cultured in vitro under axenic conditions. Both strains are cultured in ATCC® Medium 2154 (LYI Entamoeba medium) which is prepared according to the recommendations of ATCC. Culture conditions are anaerobic at 37° C. Cultures are kept in sterile 50 ml plastic tubes in a total volume of 10 ml. T. gallinae cultures are passaged regularly (in two-day intervals) in advance to maintain the strains for the study.

Quantification of total parasite number: The total parasite number is calculated separately for each tube. Therefore, the contents of each tube are centrifuged and the sediment resuspended in a given volume. A dilution of a subsample is being examined in a Neubauer counting chamber, the total number of parasites counted and the number of parasites in the respective tube be calculated.

Each treatment group consists of 3 identical replicates, i.e. 3 tubes with identical treatment (triplicate).

Five concentrations of each substance were tested against T. gallinae, namely 1 nM, 10 nM, 100 nM, 1000 nM, and 10000 nM. The total exposure time of the test compounds for the efficacy assay was 72 hours; media were renewed in intervals of 24 hours. Efficacy was measured after 24 hours of incubation.

The total T. gallinae trophozoite counts were lowest in the high-dose treated group (10,000 nM) after 24 hours of treatment for both investigated strains (see FIG. 2). In the 10,000 nM treated group, trophozoite number was 26.3% for the ATCC 30095 strain and 24.0% for the Vienna strain of the relative group NCA (=control group), respectively.

The total parasite number is calculated before a subsample is subject to counting of viable and dead parasites. For counting, parasites are stained by DAPI nucleus staining for differentiation of viable and dead parasites.

Markedly lower viable-to-dead ratios could be found in Trichomonas gallinae cultures treated with different concentrations of Z—FY—CHO compared to the control group NCA after 24 hours of treatment.

These data show the growth inhibitory and potential cytotoxic effects in vitro on cultures of T. gallinae by Z—FY—CHO.

### Example 2: Efficacy Study In Vivo

Thirty Columba livia forma domestica of both gender (2 groups of fifteen birds) were inoculated per os via a syringe with 2 million T. gallinae cultured from the Vienna strain. Initial age of the pigeons was 4-5 months. Two days later the pigeons were treated with twice daily oral administration of 5 mg/kg of Z—FY—CHO or the placebo (vehicle alone) during 4 days. The formulation was composed of 50 mg of the agent added to 1.0 ml 96 g/v % ethanol and 4 gram of prewarmed Solutol HS15. The formulation was stirred to a clear solution, then it was topped up with prewarmed distilled water to a volume of 10 ml. From this solution 0.1 ml per 100 g body weight was administered (=5 mg/kg). Thereafter the birds had twice daily a general health check as well as measurements of feed and water consumption. Fourteen days after infection the birds were euthanized and organs histologically examined. Regurgitation was evident significantly more often in pigeons from placebo group (7 of 15 pigeons, p=0.018). Regurgitation has been detected first in one pigeon from the placebo group in the evening on day of infection. One or two different pigeons of placebo group have shown this clinical signs on the following days until the end of the study. Only one pigeon of the treatment group regurgitated on study day 28 in the morning. Regurgitation was not influenced by gender, age, other clinical findings or trichomonads reisolation. No gross pathological findings in the esophagus or crop were detected during the necropsy. However, heterophilic infiltrates in the mucosa as well as in the submucous tissue and lymphocytes and plasmacells in the submucous tissue have been detected histopathologically in total eight pigeons (27%). Catarrhal erosive esophagitis and ingluvitis was significant more often detected in pigeons from the placebo group (7 of 8 pigeons with inflammatory reaction of the esophagus and crop, 88%; p=0.018). Only one pigeon of the treatment group revealed this finding. An additional finding in this study was that several pigeons suffered from coccidiosis and after treatment with Z—FY—CHO no coccidiae could be detected in these birds whereas 3 pigeons (of the 15 birds) in the placebo group still showed coccidiae. Overall, no obvious and/or statistically significant side effects were detected during the Z—FY—CHO application of 10 mg/kg per day.

Therefore the data show that Z—FY—CHO can be used to prevent invading trichomonads into the mucosa of the esophagus and crop and eventually in other organs as well as to reduce the number of coccidia in the intestine.

### LITERATURE REFERENCES

1. Amin Aziza et al., 2012 PLoS One. 7(5): 1-11, Cysteine peptidases, secreted by Trichomonas gallinae, are involved in the cytopathogenic effects on a permanent chicken liver cell culture.
2. Keeve E. Nachman et al., 2013 Environ Health Perspect 121(7); 818-824, Roxarsone, Inorganic Arsenic, and Other Arsenic Species in Chicken: A U.S.-Based Market Basket Sample
3. Berge S M et al, 1977, J. Pharm Sci. 66, 1-19, Pharmaceutical Salts
4. J-T Woo et al., 1995 Bioorganic and Medicinal Chemistry Letters 5 (14): 1501-1504, Peptidyl aldehyde derivatives as potent and selective inhibitors of cathepsin L

1. A method of treating or preventing parasitic diseases,
comprising administering a compound of formula I to a subject in need thereof, wherein formula I is:
Z-Phe-X—CHO
and wherein:
X is tyrosine, methyl tyrosine, butyl tyrosine, alanine, or leucine; and
Z represents N-benzyloxycarbonyl, or derivatives of these dipeptidyl aldehydes or a pharmaceutically acceptable salt or stereoisomer thereof.

2. The method of claim 1, wherein the compound is N-(benzyloxy-carbonyl)-L-phenylalanyl-L-tyrosine (Z—FY—CHO), or its derivatives or a pharmaceutically acceptable salt or stereoisomer thereof.

3. The method of claim 1, wherein the parasitic disease is selected from the group consisting of trichomoniasis, histomoniasis, trypanosomiasis, coccidiosis and cryptosporidiosis.

4. The method of claim 1, wherein the compound is administered to non-human mammals.

5. The method of claim 1, wherein the compound is administered to birds.

6. The method of claim 1, wherein the compound is administered together with an additional antiparasitic agent.

7. The method of claim 6, wherein said additional antiparasitic agent is selected from the group consisting of nifurtimox, arteminisin, suramin, homidium salts (ethidium, novidium), quinapyramine sulfate, diminazene aceturate, isometamidium, metronidazole, dimetridazole, carnidazole, ronidazole, toltrazuril, sulfonamides and amprolium.

8. The method of claim 6, wherein said additional antiparasitic agent is administered prior to, simultaneously with, or after administration of the compound according to claim 1.

9. The method of claim 1, wherein said compound is administered at least once a day for a period of at least 3 days.

10. The method of claim 9, wherein said compound is administered at least twice a day for a period of at least 3 days.

11. The method of claim 5, wherein the birds are pigeons.
